(19) United States
(12) Patent Application Publication
(10) Pub. No.: US 2004/0073252 A1
(43) Pub. Date: Apr. 15, 2004

(54) Blood clot filtering system

(76) Inventors: Mark Goldberg, Sharon, MA (US); Ron Mallick, Wilmette, IL (US); Lev Melinyshyn, Buffalo Grove, IL (US)

Correspondence Address: MICHAEL BEST & FRIEDRICH LLC, 401 NORTH MICHIGAN AVENUE, SUITE 1700, CHICAGO, IL, 60611-4212, US

(21) Appl. No.: 10/403,585

(22) Filed: Mar. 31, 2003

Related U.S. Application Data
(63) Continuation-in-part of application No. 10/341,283, filed on Jan. 13, 2003.
Application No. 10/341,283 is a continuation of application No. 09/788,878, filed on Feb. 20, 2001, now Pat. No. 6,506,205.
(60) Provisional application No. 60/369,362, filed on Apr. 1, 2002.

(51) Int. Cl. A61M 29/00

(52) U.S. Cl. 606/200

(57) ABSTRACT

A blood clot filtering system including an anchor which is permanently affixed in a blood vessel, and a filter which is removably attached to the anchor.

### BACKGROUND OF THE INVENTION

This invention relates generally to devices and methods for trapping blood clots and controlling embolization and some of the complications of thrombosis in blood vessels. More particularly, this invention relates to a blood filtering system that comprises two separable independent parts: a permanent anchor, and a filter removably attached to the anchor. The two parts of the system are attached in such a way that, once emplaced, the filter is continuously maintained along the central axis of the blood vessel to ensure that the filter operates at optimal efficiency. If and when it is necessary or desirable to remove the filter, it may readily be separated from the anchor and withdrawn, leaving a permanently attached anchor that does not interfere with blood flow within the blood vessel.

The presence of thrombus within the body's circulatory system presents significant health hazards, as manifested by potential acute venous thrombosis and chronic deep vein thrombosis. Acute venous thrombosis can lead to pulmonary emboli, a potentially lethal condition when an embolus travels into the pulmonary arteries. Currently, the most widespread treatment is the administration of systemic and oral anticoagulants such as heparin and coumadin, and thrombolytic agents such as TPA, urokinase and streptokinase.

Unfortunately, conventional drug therapy is ineffective or inappropriate for controlling emboli within the circulatory system of some patients. In particular, since most pulmonary emboli originate in veins of the lower limbs, pelvis or inferior vena cava, it has been recognized that life-threatening pulmonary emboli can be prevented from reaching the lungs by mechanically interrupting the inferior vena cava to filter out emboli.

Indications for introducing such filters in the inferior vena cava include:

a) Pulmonary embolism in patients with a high risk of internal bleeding, including those having surgery, anticipated surgery, recent trauma, cerebral hemorrhage or peptic ulcer disease who are not amenable to anticoagulant or thrombolytic therapy.

b) Recurrent pulmonary emboli notwithstanding anticoagulant therapy.

c) Patients showing large free-floating thrombi in the iliofemoral veins or inferior vena cava as identified with venography.

d) As prophylaxis against pulmonary emboli in older patients with high-risk conditions.

e) Disseminated thrombosis and profound thrombo-cytopenia in patients displaying heparin sensitivity.

f) Prevention of recurrent pulmonary emboli after pulmonary thrombolectomy.

In 1967-68, Eichelter and Schenk described an umbrella-like device which they introduced under local anesthesia into the femoral vein of dogs to filter emboli. Eichelter P. Schenk, W. G., Jr.: “A New Experimental Approach to Prophylaxis of Pulmonary Embolism”. rev Surg 24:455-456 (Nov-Dec) 1967; Eichelter P. Schenk, W. G. Jr.: “Prophylaxis of Pulmonary Embolism.” Arch Surg 97: 348-356 August 1968. The Eichelter/Schenk device was constructed by making longtitudinal incisions circumferentially around a segment of a polyethylene tube, placing a tube of smaller diameter inside the larger tube and flaring the end protruding beyond the linear incisions. Light traction of the inner tube while holding the outer tube stable produced an umbrella-like structure. Unfortunately, this structure included numerous apertures for trapping stagnant blood and thereby promoting highly undesirable thrombosis and potential embolization.

Eichelter and Schenk made small incisions in the right femoral veins of the groins of the dogs used in the tests with the distal portion of the catheter tied into the femoral vein and the device open at a point lying distally to the renal veins. After a number of weeks, the device was collapsed and removed through a small incision. The embolization of trapped or attached emboli upon removal of the Eichelter/Schenk device precluded use of this device in humans.

A permanent implantable vena cava filter was developed by Mobin-Uddin in 1969, and described in U.S. Pat. No. 4,540,431. This filter was intended to be introduced through an incision in the jugular vein. The Mobin-Uddin filter was an umbrella-like structure having expanding ribs carrying sharpened points at their divergent ends which impaled the wall of the blood vessel when the filter was positioned at the desired location and permitted to expand into its operative structure. The Mobin-Uddin filter had a high occlusion rate and therefore was not widely used. Finally, even if initially properly implanted, these filters could come loose and migrate to either ineffective or dangerous and life-threatening locations in the vascular system.

The present invention solves the problems inherent in the prior art devices by providing a system establishing a quick, safe, and well-centered reliable emplacement of an effective emboli filter which is secure in the vessel until it becomes desirable or necessary to remove the filter. The present invention is particularly useful for placement in the inferior vena cava. The system may also be useful in filtering clots in other areas of the vascular anatomy.

### SUMMARY OF THE INVENTION

It is therefore an object of the present invention to provide a two stage blood clot filtering system which can be quickly and safely emplaced within a blood vessel to efficiently trap emboli passing through the vessel.

Yet another object of the present invention is to provide an emplacable blood clot filtering system which, while maintaining patency, can provide either permanent or temporary protection from emboli in blood vessels.

It is yet another object of the present invention to provide a blood clot filtering system which can be emplaced through the femoral or internal jugular vein in a relatively simple procedure, during the course of which the system may be readily repositioned until optimally located in the vessel, and then positively fixed in that location for the desired, medically appropriate period.

A further object of the present invention is to provide a blood clot filtering system which can be steered through the vena cava under appropriate imaging techniques.

A still further object of the present invention is to provide an emboli or blood clot filter which, once emplaced, remains suspended along the longitudinal axis of the vessel as blood flows through the filter, minimizing endothelialization and vessel wall contact on the removable portion.

Another object of the present invention is to provide an emboli filter for emplacement in a blood vessel which can be permanently emplaced but which also can be readily removed when desired.

Yet another object of the present invention is to provide a blood clot filtering system for emplacement in blood vessels in which the patency is optimized and release of emboli into the bloodstream upon removal of the filter from the vessel is minimized.

Still another object of the present invention is to provide a blood clot filtering system including an anchor that is permanently emplacable in a blood vessel, and a removable filter attached to the anchor, in which endothelialization of the filter is minimized.

The present invention is therefore directed to a blood clot filtering system including an anchor which is permanently emplacable in a blood vessel and a blood clot filter removably attached to the anchor.

The anchor is radially self-expanding. It may be made of a metal spring wire material bent into a close zig-zag formation, with alternating zig and zag legs meeting at sharp angles at their distal and proximal vertices. At least two hooks may be provided respectively at at least two distal vertices spaced equidistantly on a circle defined by the distal vertices.

The filter preferably includes two stages which cooperate to provide enhanced clot catching. The first stage comprises a series of distally projecting legs evenly spaced about the longitudinal axis of the filter, and the second stage comprises a series of generally radially projecting legs also evenly spaced about the longitudinal axis of the filter. The first stage may be also provided with a series of flexible filamentous tethers. The filter is removably attached to the anchor by way of these tethers. Finally, the filter may include a spring-loaded jaw at its proximal end. This jaw will retain both ends of each of the tethers when the tethers are attached to the anchor, but will release one end of each of the tethers in the process of removing the filter, leaving the anchor permanently fixed in place.

In a further embodiment, it is noted that a blood clot, or thrombus, can form through a series of complex chemical events involving the activation of platelets and the coagulation pathway. Clotting is necessary in the event of trauma and stems excessive bleeding. In some situations, however, clotting can occur in a pathologic manner resulting in undesirable consequences. A common location of such pathologic clots is in the deep veins of the lower extremities and is known as deep venous thrombus (DVT). Such clots may be unstable and may fragment, resulting in portions of the original clot traveling and lodging within the lung vasculature. These clots are referred to as pulmonary emboli (PE) and may result in disruption of the process of oxygenation and lead to hypoxia and eventually death of a patient. In a subset of patients with a DVT, medical treatment using heparin to prevent a PE may fail. In this subset of patients or patients suspected of being at risk for such complications, a mechanical device should be placed between the DVT and the lungs. Such a site of implantation is commonly the inferior vena cava.

This further embodiment of the invention comprises a vena cava filter that can be disabled in place, thus avoiding the potential complications associated with the removal or retrieval of vena cava filters. The device design includes an anchor, eyelets or other filament-receiving means on the anchor and monofilament (FIG. 1′) or other filamentous material that is laced through the eyelets to form a filter configuration (FIG. 3′). The filamentous material may comprise a single strand or a plurality of strands (FIG. 2′). The filamentous material may be made from a number of materials including, but not limited to, polypropylene (such as the commercially available Prolene™ suture), PTFE, nylon, wire or other permanent and durable thread-like materials. The monofilament could be strung in a tight fashion or in another embodiment it could be loose so that it moves with the flow within the vena cava. The anchor is configured preferably in the form of a Z-stent, although other anchor means could be used (FIG. 1′). One useful anchor structure is illustrated in my copending application Ser. No. 09/788,878, filed Feb. 20, 2001.

The monofilament material preferably is strung in such a fashion as to form a cone shape, although other shapes could be used (FIG. 2′). The cone or other filament shape has a plurality of “legs” comprising loops of the monofilament. The filter may have anywhere between 3 sets of loops (six legs) up to 6 sets of loops (12 legs) forming a first stage of the filter. Of course, the filamentous shape could also be attached to the anchor in any conventional way. In one embodiment, the anchor would have at least three eyelets on the top portion and at least 6 eyelets on the bottom portion. The monofilament strand or strands are threaded through the eyelets and tethered through a ring positioned within the anchor. The ring would be preferably a radiopaque metal but could also be composed of a PTFE or PTFE-coated coated material. In this embodiment, the monofilament filter has a “star-burst” appearance (FIG. 2′). The bottom six loops comprise a first stage and the top three loops comprise a second stage.

The monofilament material is directly connected to the anchor (FIG. 3). Thus, in the simplest embodiment, as the stent anchor expands its diameter and thus shortens its length, the tension on the monofilament and the shape of the filter changes. Careful proportioning of the monofilament are required to prevent a premature halt of the expanding anchor, which could prevent contact and engagement/adherence to the vena cava wall. In another embodiment, the Z-stent is comprised of hollow elements with an elastic material that has direct or indirect connection to the monofilament loops. In an elongated but narrow diameter state, the elastic material is tense. As the anchor expands and the diameter expands, the elastic material shortens with the stent and provides less tension on the monofilament loops. In this manner, a more uniform tension and shape are provided for the filter.

In another embodiment, the Z-stent has a skin of PTFE similar to an endovascular graft or covered stent. The PTFE lining prevents endothelialization of the monofilament loops and thus enhances the subsequent removal of the strands (FIG. 3′).

The device is installed using a flexible coaxial catheter system introduced into a vein and advanced into a position slightly beneath the renal veins within the vena cava such as that described in my copending application Ser. No. 09/788,878, filed February 20, 2001. The device is deployed in a manner such that the anchor stent engages the wall of the vena cava. The monofilament is automatically centered and tension is provided by the expansion of the anchor.

The filter is not intended to be retrieved nor is it actually removed in a strict fashion. Rather, when necessary or desirable to disable the filter to permit blood flow without filtering, an instrument is inserted into the vena cava and positioned either overlying or beneath the monofilament filter. The monofilament material is cut or severed using this instrument, and the strand or strands from which it is made are pulled out thus disabling the filter. Since the material is monofilament, any endothelialization of the anchor would not incorporate the thread and it could be pulled out without disruption of the vena cava wall. Furthermore, a covered stent like anchor using a material such as PTFE would certainly prevent incorporation of the thread or anchor elements. In the case of multiple separate strands of monofilament material, each single strand would be severed and at the conclusion of the procedure, the device will be fully disabled. The anchor remains in place within the lumen of the vena cava, leaving it permanently open. In another embodiment, portions of the strands are removed from the top of the filter and the monofilament strands that wind through the anchor remain permanently attached to the anchor.

In summary, the key features of this embodiment of a vena cava filter that can be disabled include:

1. A filter that is composed of monofilament or other filamentous material preferably laced in a cone shape with a plurality of loops that comprise the “legs” and body of the filter (FIGS. 2′ and 3′).

2. A filter that is secured in place by an anchor, which in one embodiment would have features of a Z-stent with a series of eyelets to allow attachment of the filter by lacing the monofilament to the anchor (FIG. 1′).

3. Disabling the filter by severing the strands of the monofilament and pulling the strands out of the structure of the anchor.

4. A PTFE covered anchor/stent to prevent endothelializaton of the filter and removable parts (FIG. 3′).

Yet another embodiment basically involves a vena cava filter element, a separate anchor/stent with eyelets and filaments that bind the two structures in a tethering fashion and a release mechanism. Two archetypes have been described with the preferred embodiment comprising a synthesis of the two; the structural composition of the “tethered” filter combined with the release mechanism from the “filamentous” filter. Further details concerning specific form and function are outlined below:

1. Form and function of the “cylinder” which binds the filaments and allow for a safe means to sever only one side of the filaments.

2. Means for binding the filaments to the cylinder and possible manufacturing techniques.

3. More detailed discussion of the composition of the filaments.

4. Details for the descending blade and safety elements for its introduction and use.

5. The possible interaction of the filter and the anchor with attention to minimizing the filter's contact with the vena cava wall and factors for minimizing vena cava wall reaction.

6. Details concerning the eyelets and means to prevent attachment of the filaments.

7. Issues of self centering and the paradox of radial force versus floating.

8. Anchor/Stent form and function.

9. The strength of a modular design regarding engineering teams and reduced impact on changes as relates to the interaction of the components for the form and function.

10. The delivery system.

11. Clot catching during extraction of the filter.

The cylinder (FIGS. 1-3 N) is defined as the structure that resides at the top of the filter and binds the filaments in a unique and controlled fashion. The cylinder must be designed to ensure that only one filament is presented during the extraction process and that only one side of any given filament is severed. I have presented my preferred cylinder embodiment in pages 1-3 and FIGS. 1-3N. Using the mechanism for removal from the “filamentous” filter has a significant advantage over the “tethered” mechanism for removal. The tethered mechanism relies on an internal spring/friction mechanism system that could be impaired by the human response to a foreign body. In fact any “internal” mechanism could be compromised by the human response to a foreign body. The filamentous mechanism introduces the removal system from an external source and avoids the body/tissue response.

The cylinder is a hollow structure in which holes have been obliquely drilled using, e.g., a laser to provide outer wall communication with the inner cavity. The inner cavity provides a route for the “protected” side of the filament to travel and become permanently bound or fixed to the filter structure. The laser-drilled holes permit the other portion of the filament loop to become fixed to the filter. In routing the filaments in this fashion only one side of any given filament is exposed during the extraction process. The outer hole pattern would ideally be formed in a staggered fashion as to allow the presentation exclusively of an individual strand at the time of extraction. Such a pattern would present the best means to reliably sever the strands.

The filaments as they protrude through the top of the cylinder (including both the inner strands and the outer strands) are laser welded and melted into a “knot” or bound in a fixed fashion and then a hook assembly with a “cap” is preferably laser welded to the cylinder. The cap could have a rounded or flat but angled surface. Other variations are possible. The outer surface of the cylinder is a highly polished metal with close tolerance. A protective lip prevents the cutting blade from descending below the cylinder and severing the inner strands. Alternatively, the legs of the filter may serve this purpose depending on the manner in which they are attached to the cylinder. It is important that only one side of a filament or strand is severed for control of the filter and to prevent free foreign material from migrating into the lungs.

Alternatively the filament could be fixed to the distal end of the filter's leg on one end and the cylinder at the top. This would eliminate the loop aspect of my preferred design. To fix a filament to the distal end of the filter leg would require a hollow tube structure into which the filament could be wedged.

Another alternative is the staggering of eyelets. The analog I use is that three points define a plane and compared the stability of a chair with four legs versus a stool with three legs. Also of importance, is that the eyelets and filaments should be positioned off center or skewed relative to the “true” legs of the filter element. This will provide additional confidence that the legs of the filter element do not become bound up by the filaments. By also positioning the end of the legs for the filter element beneath the eyelets, it should also provide a greater margin of safety. If the legs of the filter are well below the looping of the filaments, then binding of the legs should be avoided.

The removal procedure would require a wire snare that would engage the hook at the top of the cylinder. An outer catheter/sheath would be advanced toward the cylinder body. This could be facilitated by placement of (e.g., platinum) markers on the outer catheter, cutting blade catheter and cylinder as depicted on page 2-1. A cutting blade catheter would be advanced over the guide wire snare but within the outer sheath for safety purposes. Ideally, the cutting blade catheter would have a portion of its outer surface contact the side of the outer catheter to improve stability and safety, and most importantly, to provide axial rigidity. One such illustration of such a configuration is seen on page 2-1. Other configurations are possible. The cutting surface could be provided in a flat “cookie cutter” design; alternatively serrated blades with a continuous cutting surface similar to my valvulotome patents could be used. In its preferred embodiment, the blade would engage one strand at a time to improve the effectiveness of cutting the filaments. As mentioned earlier in this discussion, a staggered configuration of the filaments as they enter the outer wall of the cylinder could provide a means for achieving this goal. The close tolerances for the inner diameter of the sharp cutting blade and the outer wall of the cylinder would be created to provide a “scissors” like mechanism for severing the filament. The protective lip or proximal legs of the filter would provide a “stop” and prevent the injury to the other side of the strand.

I have explored different configurations and shapes for the cap to the cylinder in order to enhance the positioning of the cutting blade relative to the cylinder and filaments. It is not yet clear whether one embodiment has an advantage over another. FIG. 1N depicts two different ways in which the cap to the cylinder could be shaped.

After the strands have been severed, then the blade catheter would be removed and the filter with filaments would be pulled up into the outer catheter as the filaments would freely traverse the eyelets of the anchor. Ideally the filaments would be a thin material with a smooth non-adherent surface. One material that would facilitate this process is PTFE. I have been told that PTFE strands or “fibers” can possess a very significant strength advantage over polypropylene for any given diameter. PTFE also has a significant advantage over wire filaments relative to a given diameter when attempting to sever it. A PTFE coating could additionally be provided to the eyelets to enhance a low coefficient for friction and improve the extraction of the filament through the center of the eyelet. Also of possible benefit would be the use of drug molecules bound to the surface of the anchor, eyelets and filter to prevent intimal hyperplastic response similar to a coronary stent.

The positioning of the eyelets has a significant impact upon the form and function of the filter. Since it is a modular system, many elements can be modified. Some of these modifications can adversely impact upon the French size of the deployment system. I prefer the distal legs to overlay the anchor. This will probably result in a slightly larger deployment package, but has many advantages. The distal legs could be positioned within the anchor to interface and contact with a pad preferably made from PFTE. This would prevent the filter legs from contacting the vena cava wall. This would help extend the theoretical limits for removal of the filter to the lifetime of the patient. The point, however, is to “over engineer” the removal mechanism and provide the widest margin of safety that could not be exceeded by a doctor regardless of limits agreed to with the FDA. Another advantage of positioning the filter to overlap the anchor can be seen with the relative position of the filaments to the distal end of the filter legs. If the filter leg(s) extends into the anchor and if the eyelets are positioned above the distal end of the filter legs, the filaments would not bind or entrap the legs of the filter. This provides an enhanced margin of safety and could prevent a faulty placement of the filter and also prevent tilt.

Tilt is a significant issue for a filter design. There are several parameters one can manipulate in order to prevent or limit tilt. Once issue concerns the radial force of the legs outwards against the vena cava wall. This has been traditionally used to help with tilt. Obviously, tilt is still observed even when significant radial forces are used to center the filter. Although, conceivably, an increased radial force could be more evenly distributed through the anchor and thus better tolerated than a conventional filter with hooks, such as the Greenfield filter, an alternative may be to use less force. It is possible that the force derived from the flow in the vena cava may help “float” the filter into a centered position and may better serve in a structure with a constantly changing diameter and shape such as the vena cava.

The constantly changing diameter and shape of the vena cava in part makes placement of a device a difficult enterprise. I believe that a stent may present a better stable means than a hook for a filter. I think a very important issue in regards to tilt is the deployment system and indirectly because of the deployment system—the speed at which the filter is deployed. For example, the Greenfield filter when deployed springs out and engages the vena cava wall when the distal aspect of the hooks clears the deployment catheter. If the vena cava wall is constantly changing its configuration, then a quick deployment may result in one leg contacting at an angle and which then may affect the orientation of the remaining legs. A means for slow deployment may help allow a more symmetric engagement of the vena cava wall. This is in part why the overlap of the filter element with the anchor offers an advantage over other configurations.

The deployment system I have envisioned would involve a slow retarded release of the filter assembly. From a femoral approach this is not a complicated process. An internal jugular approach would require a different means for a slow release. A slow release system, however, would not necessarily make the deployment a more difficult process for the physician.

The stent/anchor design should be optimized for the smallest foot print that still achieves the goals of providing a secure attachment to the vena cava, inhibiting tilt of the device, and preventing migration of the filter. A conventional stent could be used or modified but I think that a separate approach may be more meritorious.

In summary, I have presented a concept for a removable vena cava filter that is characterized as a filter element linked to an anchor by filaments that may be severed for the extraction of the filter element. Revision of any component of this system has minimal impact upon the function and form of the other modules. For example, revision of the cylinder that comprises the top of the filter element and provides exposure of a single strand of the filament loop would have little effect on the anchor design. This is a significant advantage over conventional designs in which modification of the hooks could significantly impact on the geometry and design of the legs.

### DESCRIPTION OF THE DRAWINGS

The objects and advantages of the present invention will be described with respect to the following figures in which:

FIG. 1 is a perspective view of the filtering system of the present invention in which the filter and anchor of the system are separated for illustration purposes;

FIG. 2 is an enlarged cross-sectional view of the jaw at the proximal end of the filter of FIG. 1;

FIG. 3 is perspective view of the anchor of the filtering system of the present invention;

FIG. 4 is an enlarged view of one of the curls at a proximal vertex of two zig-zag legs of the anchor showing a tether filament passing through the loop in the curl;

FIG. 5 is a perspective view of the assembled filtering system of the invention showing the filaments of the filter tethers passing through loops at alternating proximal vertices;

FIG. 6 is a perspective view of the assembled filtering system of the invention showing the filaments of the filter tethers passing through adjacent pairs of proximal vertices;

FIG. 7 is an elevation view of a flexible introducing catheter partially cut away to show the assembly of the filter and anchor collapsed radially and positioned in the catheter;

FIGS. 8A-8G illustrate diagrammatically the steps in emplacement of the filter system of the present invention in a blood vessel;

FIGS. 9A and 9B illustrate a removal catheter including an umbrella which may be retracted and deployed from the catheter;

FIG. 10 is a cross-sectional view of the removal catheter of FIGS. 9A and 9B, including a snare handle mounted to the catheter;

FIG. 11 is an enlarged cutaway partial view of the end of the snare catheter abutting the jaw at the proximal end of the filter;

FIG. 12 illustrates an alternative design of the jaw depicted in FIG. 2 in which the proximal hook is replaced by a ball;

FIGS. 13A and 13B illustrate the capture of a ball at the proximal end of the jaw and the application of a distally directed force for releasing the tether filaments and removing the filter from a blood vessel in which it was previously deployed; and

FIG. 14 is a diagrammatic representation of a blood clot filter emplacement kit containing the filtering system of the present invention.

### DESCRIPTION OF THE INVENTION

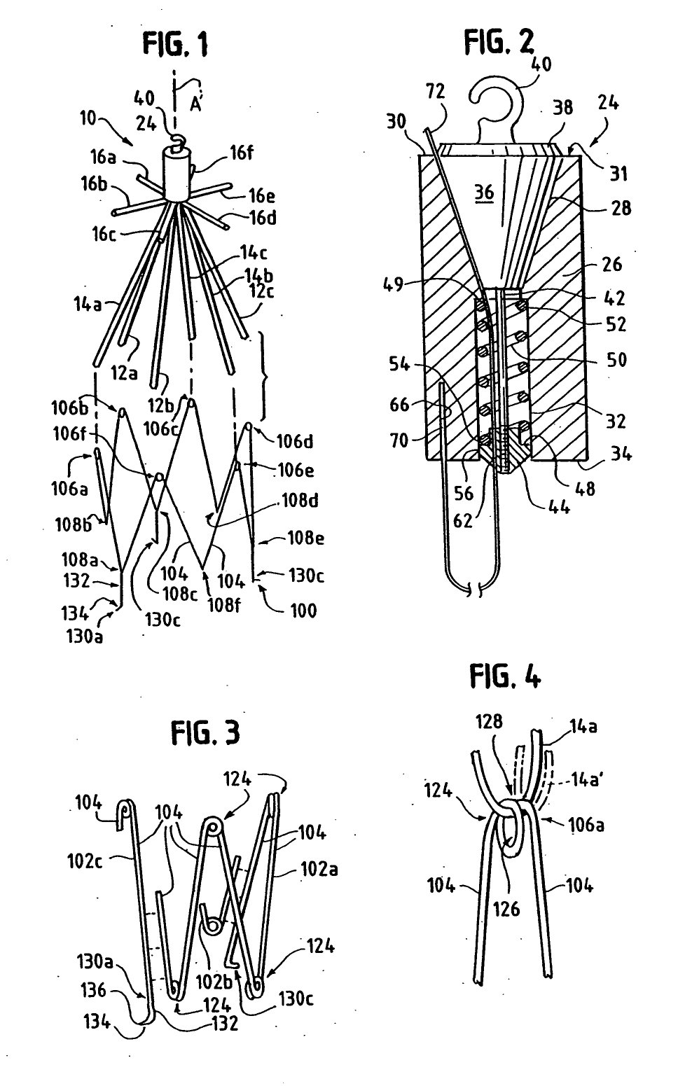

Turning first to FIG. 1, the blood clot filtering system 8 of the present invention is shown. The system includes a two stage filter 10 and an anchor 100. Two stage filter 10 comprises a series of distally projecting legs 12a-12c, evenly spaced about the central axis A of the device, constituting one portion of the first stage of the filter. While three distally projecting legs 12a-12c are illustrated, and constitute a preferred embodiment, four, five, six or more generally evenly spaced legs may be used. Also, the legs are shown in their fully open, non-in vivo position, at an angle of about 12° to longitudinal axis A, which is preferred. However, distally projecting legs 12a-12c may be at any angle ranging from about 2° to about 22° to axis A when at rest, before placement in an introducing catheter or emplacement in a blood vessel.

Distally projecting legs 12a-12c are made of a spring-like material which gives each leg rigidity along its longitudinal axis while permitting it to flex laterally, thereby enabling the filter to assume a fully closed configuration (FIG. 7) in which the legs are moved radially inward until they abut or nearly abut each other adjacent axis A of the filter. The distally projecting legs may be made from metal, for example, from stainless steel, nitinol, or Elgiloy® alloy (available from Egiloy L. P. of Elgin, Ill., USA). In the illustrated embodiment, the distally projecting legs are made from stainless steel wire, and have a diameter of about 0.008 to 0.012 inch and preferably a diameter of about 0.010 inch.

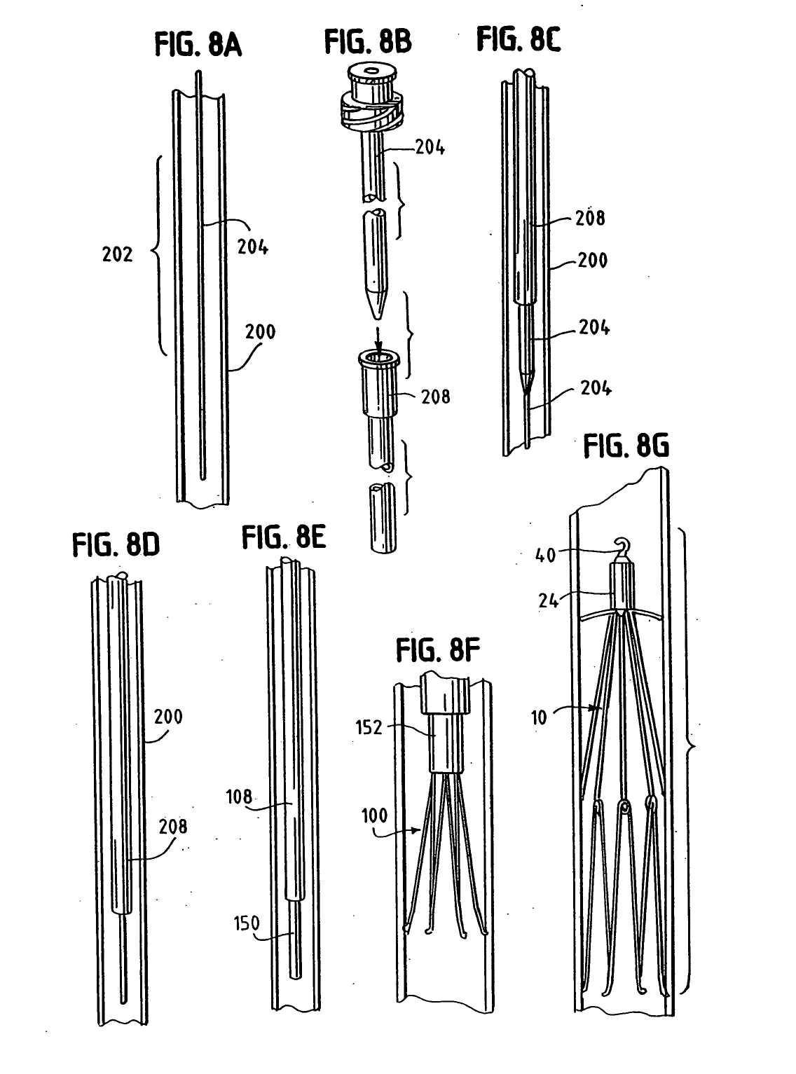

The filter is intended to be in a fully closed configuration as it is inserted into or removed from a blood vessel, as described in more detail below. When the filter is deployed in a blood vessel (FIG. 8G), distally projecting legs 12a-12c will be flexed inwardly to a degree intermediate between the fully open and fully closed positions.

The first stage of the filter also includes a series of flexible filamentous tethers 14a-14c which, in the illustrated embodiment, are located between adjacent pairs of distally extending legs 12a-12c. Tethers 14a-14c may be round or flat and are made of a flexible, elastic material such as nitinol or stainless steel, or of nylon monofilament or other synthetic filamentous material. In the illustrated embodiment, the tether filaments are preferably flat and made of nitinol having a width of about 0.005 inch.

These tethers, which are attached to the filter and loop back from the anchor, providing filament loops, serve at least three purposes. The first is the attachment of the filter to the anchor in such a fashion that the filter will be centered and generally continuously maintained along the central longitudinal axis of a vessel in which the blood clot filtering system is deployed, insuring that the filter operates at peak efficiency. Second, the tethers permit the filter to be separated from the anchor when desired, so that the filter may be removed from the vessel. Finally, the filament loops of the tethers are an important feature of the first stage of the filter cooperating with legs 12a-12c. The tethers thus aid in first stage filtering by increasing the surface area coverage of the filter to improve the clot catching ability of the first stage of the filter which minimizes the likelihood of pulmonary embolization.

Filter 10 also includes a series of generally radially projecting legs 16a-16f which comprise the second stage of the filter. These legs are spaced generally evenly about the longitudinal or central axis A of the filter. Preferably, each of second stage legs 16a-16f is located in a plane defined by axis A and the proximal leg which generally bisects the interstice between each of distally projecting legs 12a-12c and its adjacent tethers 14a, 14b, and 14c. Although six such radially projecting second stage legs are shown in the illustrated embodiment, the number of legs may range from about 6 to 12.

In the illustrated embodiment, when the filter is in its fully open position, second stage legs 16a-16f extend proximally at an angle of about 70° to the central axis A of the filter, which is preferred. However, the radially projecting second stage legs may be at an angle from about 50° to 90° to central axis A. As in the case of the distally projecting legs, radially projecting second stage legs 16a-16f are made of a spring-like material which gives each leg longitudinal rigidity while permitting it to flex laterally. This enables the filter to assume a fully closed configuration (FIG. 7) in which the legs may be moved together until they abut or nearly abut each other adjacent axis A of the filter. As explained above, the filter is intended to be in this closed configuration as it is inserted or removed from a blood vessel.

The radially projecting second stage legs will be flexed inwardly to a degree intermediate between the fully open and fully closed positions when the filter is deployed in a blood vessel (FIG. 8G). The radially projecting second stage legs may be made, for example, from stainless steel, nitinol or Elgiloy® alloy. In the illustrated embodiment, these legs are made from round stainless steel wire having a diameter of about 0.008 to 0.012 inch. Flat or round wire may be used, although round wire is preferred.

The first and second stages of filter 10 cooperate to provide enhanced clot catching. Thus, the first stage encounters and captures most clots while the second stage traps any emboli that might slip by the first stage, preventing emboli from proceeding beyond the filter.

Filter 10 also includes a spring-loaded jaw 24 having an open hook 40 at its proximal end. Jaw 24 is described in more detail in the discussion of FIG. 2 which follows.

FIG. 2 is an enlarged cross-sectional view of jaw 24 including a jaw body 26 having a truncated conical cavity 28, a proximal end 30, a distal end 34, and a bore 32 extending from the truncated distal end of cavity 28 to the distal end 34 of the jaw. A top truncated conical member 36 is shaped and sized to fit in conical cavity 28 with a portion 38 of the conical member protruding beyond the proximal end 30 of the body of the jaw encircled by an annular shoulder 31 at the proximal end of the jaw body. Hook 40 is attached to the protruding portion 38 of the conical member, centered on the longitudinal axis of the jaw.

A rod 42 is affixed to the distal end of conical member 36 and extends distally therefrom, along the central axis of the conical member. A cap 44 is affixed to the distal end of rod 42. Cap 44 is cylindrically shaped and sized to fit snugly but slideably within bore 32, and has a smooth conical distal tip 46 and an annular shoulder 48 at its proximal end. Conical cavity 28 opens at its distal end into cylindrical bore 32. Since the truncated distal end of the conical aperture has a diameter less than that of the cylindrical bore, an annular shoulder 49 is formed at this intersection. Encircling rod 42 is a compression spring 50 with the proximal end 52 of the spring resting on annular shoulder 49 at the intersection of the conical aperture and the cylindrical bore and the distal end of the spring 54 resting on shoulder 48 of the cap. Thus, compression spring 50 is compressed and confined in bore 32 between shoulders 48 and 49, maintaining conical member 36 in cavity 28. In a preferred embodiment, silicone grease may be placed in bore 32 to minimize sticking in the jaw over time. Alternatively, the inner surface of the bore and/or the outer surface 56 of the cap may be coated with polytetrafluoroethylene (Teflon®) or another low resistance or surface-modifying material which minimizes sticking.

Cylindrical cap 44 includes longitudinal bores 62 generally evenly spaced around rod 42 that pass through the cap. The number of bores 62 correspond to the number of tethers in the filter. Thus, although one throughbore is shown in the cutaway representation of jaw 24 in FIG. 2, in the illustrated embodiment of the invention there are three longitudinal throughbores 62 at roughly 120° spacings about the central axis of the cylindrical cap corresponding to tethers 14a, 14b, and 14c. Additionally, the body of the jaw includes a like number of blind longtitudinal bores 66 extending proximally from the distal end 34 of the body member and evenly spaced about the longitudinal axis of the jaw. (As in the case of throughbores 62, only one blind bore is shown in the cutaway representation of jaw 24).

FIG. 2 shows one of the three tethers (14c) which, for illustration purposes, is foreshortened. One end 70 of the filament of tether 14c is fixed in bore 66 by conventional means such as swaging or laser welding. After being passed through the anchor, the tether filament is passed through bore 62 past the individual coils of compression spring 50, and out along the surface of conical cavity 28 with the distal tip 72 of the tether filament at the proximal end 30 of the body of the jaw. Conical member 36 which is firmly resiliently seated in cavity 28 under the biasing force of spring 50 thus locks the tether filament between the abutting surfaces of cavity 28 and conical member 36. When a force is applied proximally to hook 40 while the jaw is restrained along shoulder 31, spring 50 is compressed, unseating conical member 36 and causing a gap to open up between the two abutting surfaces, releasing or unlocking tether 14c. When it is released, the tether is free to pass back out through the coils of the spring and bore 62, so that the two stage filter 10 may be detached from anchor 100 and withdrawn proximally from the vessel in which it was emplaced. Jaw 24 in cooperation with tethers 14a, 14b, and 14c therefore makes it possible to simply and efficiently separate filter 10 from anchor 100, in a procedure as described below.

Anchor 100 is self-expanding and includes a series of joined wire segments 102a, 102b, and 102c (FIG. 3) which are each bent into a close zig-zag formation, with alternating zig and zag legs 104 meeting at sharp angles at their vertices. As shown in the figures, the vertices preferably present a rounded, rather than a pointed tip. While the number of zig and zag legs and hence vertices may vary, in a preferred embodiment, as illustrated in FIG. 1, there are twelve zig and zag legs, resulting in six proximal vertices 106a-106f, and six distal vertices, 108a-108f In practice, the number of zig and zag legs and hence vertices may range from six to eighteen. As illustrated in FIG. 3, anchor 100 may be made of a series of separate wire segments which are spot or laser welded together as indicated in dashed lines. Of course, the anchor may be made of a single piece of wire, if desired.

The wire or wires from which filter 100 is made are a metal spring wire material, such as stainless steel or nitinol. In the illustrated embodiment, stainless steel wire is used which is presently preferred. The use of spring material and the zig-zag structure permits the anchor to be squeezed radially together, so that it takes up a minimal amount of space radially, to facilitate emplacement of the anchor, as described in more detail below.

Spring hinges or “safety pin curls” 124 are formed at each of the vertices, 106a-106f and 108a-108f. These spring hinges are preferred, but may be dispensed with in a less preferred embodiment of the invention. The spring hinges make for an enhanced radially outward spring force which improves retention of the anchor in a blood vessel. Also, it is preferred that alternate vertices be offset from each other in order to minimize interference between adjacent hinges when the anchor is in the fully closed position. This offsetting affects the manner in which the safety pin curls contact each other when the anchor is collapsed into the introducing catheter. If all pairs of zig zag legs were equal in length, the curls would “stack up” and take more radial space when collapsed. By alternating the leg lengths, and therefore the positions of the vertices, the curls are staggered and thus require less radial space when the anchor is in the fully closed position.

Three hooks 130a, 130b, and 130c are provided respectively at distal vertices 108a, 108c, and 108e. At least two such hooks must be present, and preferably from two to six hooks will be used. In all cases, the hooks are preferably spaced equidistantly along the circle defined by the distal vertices. In the illustrated embodiment, the hooks are formed from protruding end portions of the wire segments from which the anchor is made. Each of the hooks includes a longtitudinal portion 132 and a radial portion 134. Radial portion 134 is preferably sharpened to a point 136 (FIG. 3). Thus, when the anchor is emplaced in a blood vessel and permitted to expand outwardly under the spring force produced at the vertices of the zig-zag segments, the radially outward force seats and retains the anchor in place. Additionally sharpened points 136 engage the vessel wall, further fixing the anchor in place.

Surface modifiers for reducing or preventing endothelialization, such as Rapamune® (rapamycin) which is available from Wyeth-Ayerst Laboratories Division of American Home Products or Taxol® (paclitaxil) which is available from Bristol-Myers Squibb, may be applied to every part of the filtering system except the anchor, including the filter legs, tethers and jaw. Such surface modifiers might not be applied to the anchor because limited endothelialization on the anchor surfaces is desirable to cover those surfaces thereby enhancing anchoring and minimizing contact between the blood flowing past the anchor and the metal from which the anchor is formed.

In assembling the filter to the anchor, tethers 16a-16c are passed through the three vertices 106a, 106c, and 106e. When safety pin curls 124 are used, it is important, as illustrated in the enlarged partial view of FIG. 4, that the tethers (e.g., tether 14a in FIG. 4) pass through the loops 126 of the safety pin curls, and not in the space 128 between the abutting coils, as shown in the broken line representation of the tether 14a. In the latter case, the filament could be pinched between the abutting coils, which could interfere with separation of the filter from the anchor.

In order to clarify the way in which the filter is assembled to the anchor by way of the tethers, the assembled system is shown in FIG. 5 with the anchor fully expanded and with its three hooks 130a, 130b, and 130c resting on a horizontal surface 132. The filter is lowered somewhat with respect to the anchor to cause the tethers to balloon outwardly for illustration purposes. Thus, it can be seen in this figure that the filaments of tethers 14a, 14b, and 14c extend from jaw 24 respectively through the curl loops at vertices 106a, 106c, and 106e, and back up into the jaw to be removably held therein, in the manner described above with respect to the structure and operation of the jaw. When the system is deployed in a vessel, the anchor is compressed radially inward as it abuts the walls of the vessel, as are the legs of the filter. In this in vivo configuration, the filaments of the tethers will be elongated and drawn more closely together, generally as shown in FIG. 8G, which is discussed below.

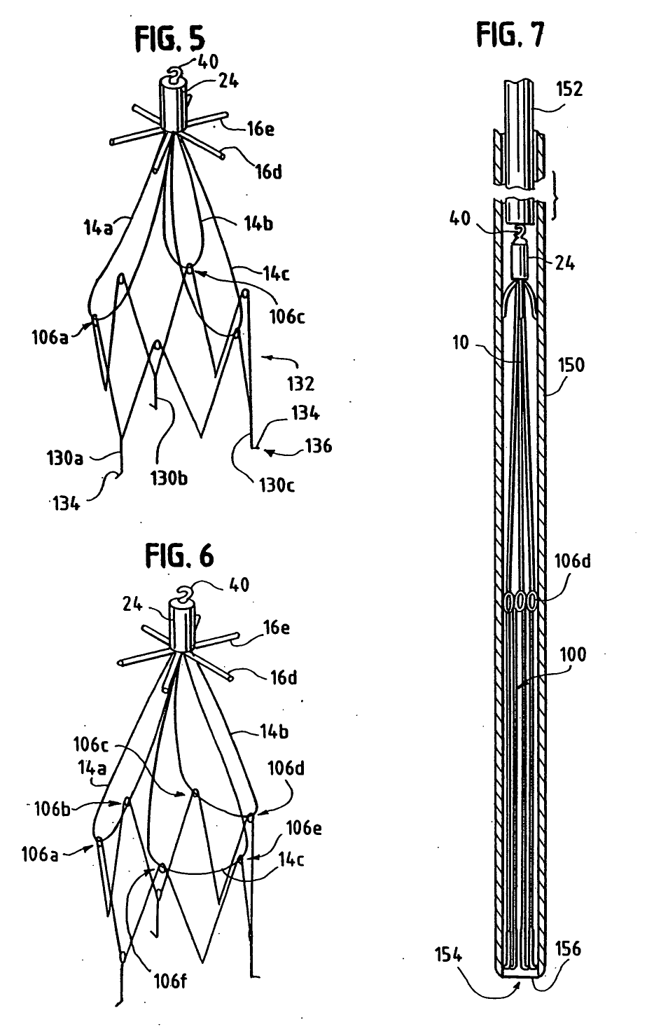

FIG. 6 shows an alternative way in which the filter may be assembled to the anchor by way of the tethers. In this figure, tether filaments 14a, 14b, and 14c pass from the jaw through adjacent pairs of curl loops at adjacent vertices 106a and 106b, 106c and 106d, 106e and 106f, and back to the jaw.

Before deploying the filter system of the present invention, the assembly of the filter and anchor are collapsed radially and placed in a flexible introducing catheter 150, as illustrated in FIG. 7. In the illustrated embodiment, catheter 150 is shown, cut away in order to make it possible to view the assembled filter and anchor in the catheter. Also shown in this figure is a pusher 152, which is used to deploy (by pushing) the attached catheter and anchor from the annular aperture 154 at distal end 156 of the catheter when the catheter is positioned at the location within the blood vessel at which it is intended to be used.

Actual emplacement of the filter system of the present invention is shown in FIGS. 8A-8G which illustrate an internal jugular approach. It is important to note that this system can be adapted to a femoral approach as well. Turning first to FIG. 8A, a portion of the vena cava vessel 200 is illustrated diagramatically at the desired implant site 202. As can be seen in this figure, a guidewire 204 has been inserted in the vena cava so that it extends beyond the implant site. Next, as shown in FIGS. 8B and 8C, a conventional dilator 206 and sheath 208 assembly is passed over the guidewire and advanced therealong until the sheath and dilator reach beyond the implant site (FIG. 8C).

Next, dilator 206 and guidewire 204 are withdrawn and sheath 208 is flushed with heparinized saline to prevent thrombus formation in the sheath. A venacavogram is then obtained by injecting a contrast medium through the sheath 208 so that the position of the sheath can be adjusted to optimize the later positioning of the anchor and filter. This leaves sheath 208 deployed on guidewire 204, as illustrated in FIG. 8D.

Now, introducing catheter 150, with the preloaded filter/anchor assembly as illustrated in FIG. 7, is flushed with heparinized saline, and then passed through sheath 208, until the introducing catheter protrudes beyond the end of the sheath, as illustrated in FIG. 8E. Now, pusher 152 (FIG. 7) is inserted until it meets the loaded filter system and held stationary in that position while the introducing catheter is slowly withdrawn, which deploys first the anchor, as shown in FIG. 8F, and then the entire filter system 8, as illustrated in FIG. 8G.

As noted above, one advantage of the present invention is that it makes it possible to easily remove the filter, if and when desired. As will be explained in greater detail below, removal generally entails: 1) restraining shoulder 31, 2) snaring hook 40, 3) pulling up upon the hook to open jaw 24 and release the tethers, making it possible to separate the filter from the anchor, and 4) withdrawing the filter from the vessel, leaving the anchor in place. When the filter is removed, the design of the filter, particularly the longitudinally rigid, smooth surfaced legs of the first and second stages of the filter, act as pins which minimize contact and resistance during withdrawal. Also, while the filter is held in place by the firmly attached anchor, the filament legs have little if any contact with the wall of the vena cava.

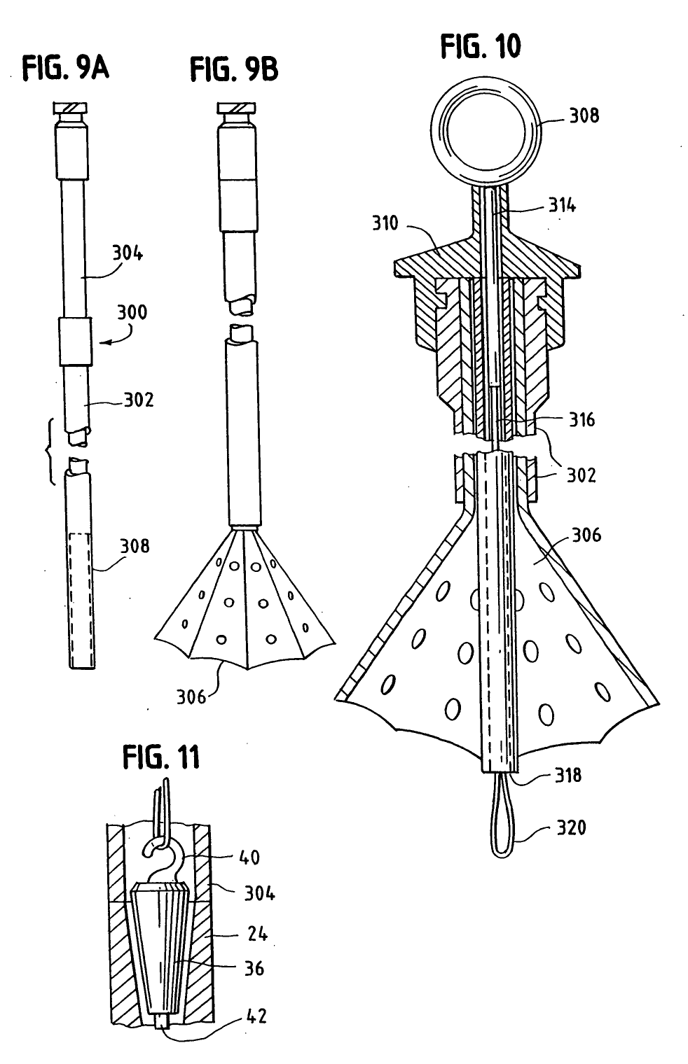

For example, as shown in FIGS. 9A and 9B, a snare or removal catheter 300 is shown having an outer sleeve 302 and an inner hollow umbrella shaft 304. An umbrella 306 is collapsed and resting in the distal end 308 of the removal catheter. Thus, when the sleeve 302 is retracted, umbrella 306 is deployed and opened, as in FIG. 7B.

Turning now to FIG. 10, further details of the snare catheter are illustrated. As shown in this figure, the snare catheter includes a pull ring 308 at its proximal end, mounted to a snare handle 310 which is fit onto the proximal end 312 of the snare catheter. The pull ring has a distally directed shaft 314 with a snare wire 316 which is attached at one end to shaft 314, and passes down through catheter and out of its distal end 318, where it forms a snare loop 320 before it passes back up through the shaft and is attached at its other end to shaft 314. Snare loop 320 may be angled up to 90° from the longitudinal axis of the snare catheter to make it easier to use in snaring hook 24 (as discussed below). Also, the size of the snare loop may be made adjustable as needed.

Thus, when it is desired or necessary to remove a previously emplaced filter, the removal catheter is passed down through the vessel in which the filter system of the invention is emplaced until snare loop 320 latches onto hook 40, with the distal end 318 of shaft 304 abutting the annular shoulder 31 of the jaw (FIG. 11). Umbrella sleeve 302 is then retracted to deploy umbrella 306 in the vessel. Once the snare loop, sleeve and umbrella are in this position, the user pulls distally on the snare ring to retract snare loop 320, pulling on hook 40, and releasing the tethers so that the snare catheter and filter may be withdrawn through the vessel leaving the anchor in place. Umbrella 306, which is optional, will catch any clots which may be freed during the procedure, which otherwise could cause the clinical manifestation of a pulmonary embolus. The umbrella should be permeable to prevent obstruction of normal blood flow. This may be achieved, for example, by providing holes 307, as shown in the illustrated embodiment and/or the umbrella may be made of a fine mesh material (not shown).

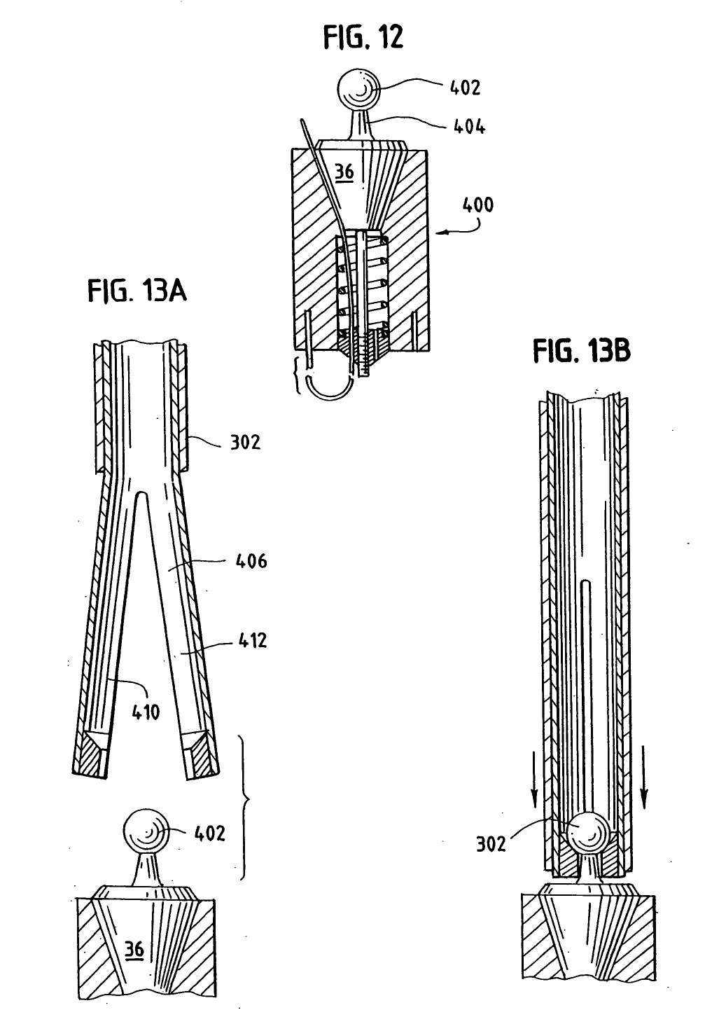

An alternate embodiment of the invention as it applies to the removal of the filter system is illustrated in FIGS. 12 and 13. Thus, a jaw 400 is illustrated in FIG. 12. As is apparent from FIG. 12, this jaw corresponds to that of FIG. 2, except that hook 40 has been replaced by a ball 402 attached to the distal end of top conical member 36 by way of a pedestal 404. A locking sleeve 406, as shown in FIG. 13A, is provided at the end of the removal catheter. The locking sleeve is shown in this figure in its extended position, with a pair of clasping jaws 410 and 412 in their open position, juxtaposed just above ball 402 of the jaw. Thus, turning to FIG. 13B, locking sleeve 406 has been moved to its fully retracted position, withdrawing the clasping jaws 410 and 412 into the catheter, causing them to pivot radially inward and to lock upon ball 402. As in the above discussion of FIGS. 10 and 11, the catheter is then withdrawn, causing jaw 400 to release the tethers so that the filter may be removed from the blood vessel.

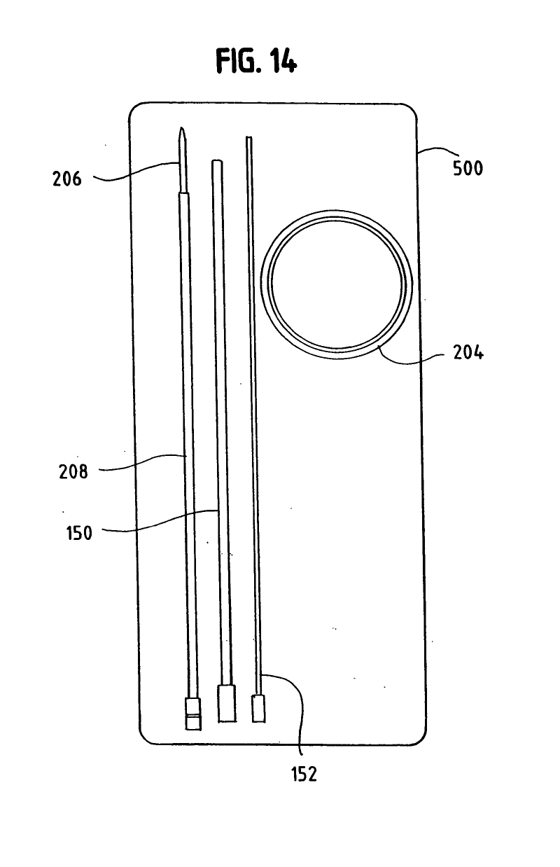

Finally, a blood clot filter emplacement system is illustrated diagrammatically, in kit form, in FIG. 14. This figure includes a container 500, containing an introducing catheter 150 with a preloaded filtering system, generally as illustrated in FIG. 7, in which the filter is oriented for emplacement from above through an upper central vein which could include the internal jugular, subclavian or brachial vein. The position of the filter in the introducing catheter could be reversed for emplacement from below, through the femoral vein. Container 500 also includes a sheath 208 with a dilator 206 contained therein, a coiled guidewire 204, and a pusher 152. The blood clot filtering system of the present invention may be conveniently provided to a user in this kit form to facilitate the emplacement procedure.

There have been described herein a blood clot filtering system and a method for its use free from the shortcomings of the prior art. It will be apparent to those skilled in the art that modifications may be made without departing from the spirit and scope of the invention. Accordingly, it is not intended that the invention be limited except as may be necessary in view of the appended claims.

What we claim is:

1. A blood clot filtering system comprising:
an anchor including means for permanently emplacing the anchor in a blood vessel; and
a blood clot filter removably attached to the anchor.
